(19) United States
(12) Patent Application Publication
(10) Pub. No.: US 2002/0188242 A1
(43) Pub. Date: Dec. 12, 2002

(54) Method and invention for the treatment of diseases and disorders of the cervix

(76) Inventor: Allan Wu, Cincinnati, OH (US)

Correspondence Address: ALLAN YANG WU, 5197 Concord Crossing Drive, Mason, OH, 45040, US

(21) Appl. No.: 09/878,190

(22) Filed: Jun. 12, 2001

(51) Int. Cl. A61N 1/30

(52) U.S. Cl. 604/21; 606/34; 607/138

(57) ABSTRACT

A method and device for delivering therapeutic agents to the cervix. The method utilizes a diaphragm with an electrically conductive surface that is placed atraumatically over the outer surface and inner os of the mammalian (animal or human) cervix. The agent is delivered to the diaphragm after which the conductive surface of the diaphragm may be charged by a power source to deliver a electrophoretic field to force the agent deep into cervical tissue and cells. The diaphragm may be used with or without the aid of external or internal leads. A unique intrauterine lead designed in this patent may also be used with the diaphragm to enhance delivery of therapeutic agents.

### BACKGROUND OF THE INVENTION

Cervical dysplasia and cervical carcinoma in-situ has been estimated at an occurrence of 500,000 new cases per year in the United States and is a disease commonly found in young women of childbearing age (Schoell 1999). Furthermore, cervical cancer is the second most common female malignancy and has a 5 year survival rate of 40% (Parkin 1993). Treatments for this disease usually entails either single or repeated treatments of locally destructive modalities such as cryotherapy, cold coagulation, diathermy loop, electrocautery or laser ablation. Though effective, these treatments, unfortunately, can be detrimental to fertility and normal childbearing. In the attempt to encircle all abnormal tissue and the transformation zone of the endocervical lining, the cervix may loose the ability to withstand the weight load of a normal gestation towards term. A solution to the incompetent cervix is surgical cerclage, which carries high risk of failure, infection and detriment to future fertility. Additionally ablative techniques may leave behind residual microscopic abnormal tissue. This predicament is usually addressed by additional ablative treatments, further increasing damage to the cervix.

Thus far, our knowledge of cervical carcinoma pathogenesis is limited. It has been well documented that human papilloma virus infection is the greatest risk factor predisposing women to cervical cancer (Eriksson 1999). However, only 30-40% of cervical cancers can be explained by HPV infection alone (Hasuo 1993). Additional mutations and/or deletions in the human genome (particularly at 11q13 and 3p) seem to be necessary for malignant and metastatic progression (Jesudasan 1995). This presents the unique opportunity of gene replacement therapy by delivery of lost tumor suppresser genes to treat and prevent cervical neoplasia. Already preliminary studies introducing recombinantly engineered vectors to deliver tumor suppresser genes and inhibitors of oncogenes in vitro have been explored with promising results (Hamada 1995). Delivery of these gene-type therapies in vivo has been attempted through use of lipofection, direct injection or systemic exposure with viral delivery (Mitchell 1996). Lipofection, however, runs the risk of non-selectively transfecting non-cervical tissues such as the vaginal epithelium and the intrauterine lining. Additionally, intracellular penetration via direct injection is limited by the accuracy of the injection. Moreover the process of introducing a syringe needle through diseased cervix and normal tissue has the risk of seeding and spreading micrometastases, as in the case of malignant and premalignant neoplasias. Viral delivery of gene therapeutics for cervical cancer, as with other diseases, can be inactivated by a response from the host-immune system, thus limiting the efficacy and ability to provide repeated treatments if required. This may be circumvented with immunosuppressants, however, this strategy places patients at further risk of undesired side-effects and hospitalizations secondary to immune suppression. Central to the success of gene therapy and/or vaccination for malignant and pre-malignant cervical disease is an efficacious and site-specific method for delivery of therapeutic agents leaving minimal or no trauma to the cervix.

Using cervical electroporation in situ and in vivo has not been attempted, documented or reported. The device presented below offers, but is not limited to, two novel distinct advantages: 1. an alternative to destructive ablative techniques, 2. an adjuvant or therapy in itself for eliminating residual cancerous and pre-cancerous cervical lesions after standard ablative techniques and 3. the possibility of repeated treatment with non-viral recombinant material.

In this invention we describe a new therapeutic device that could be used as either an alternative to standard ablative techniques therapy or as an adjuvant to minimize cervical trauma and the associated detrimental effects on fertility secondary to cervical trauma. The device is a cervical cup/glove when in contact with the cervical and endocervical surface, is capable of delivering therapeutic agents into dysplastic/malignant cells via an electric pulse. This device offers the possibility of producing cures for cervical neoplasms in addition to offering adjuvant therapy to standard ablative therapy.

### TECHNICAL FIELD OF THE INVENTION

The field of electroporation has come to be understood as the use of electrical currents or fields to force charged particles or drugs into mammalian cells (Zimmerman 1976). The exact mechanism whereby electroporation (also known as iontophoresis) works has not been fully elucidated. Theoretically it is thought that the electrical field induces micropores that allow the passage of charged particles into the cell (Zimmerman 1976). The seminal studies on electroporation involve in vitro cellular model systems. This field has now advanced to the in vivo stage where several catheters and devices, both invasive and non-invasive, are being designed and developed for various therapeutic applications (Okino 1992). To date, electroporation systems, catheters and devices have been designed for endovascular or endoluminal applications such as treatment of vascular disease and alimentary cancers. A thorough search of the medical and life sciences literature and patent database has not revealed any tailored device specifically for the use of cervical neoplasias.

### DISTINCTIONS FROM PRIOR ART

In U.S. Pat. No. 5,749,845 an electroporation device was elaborated for the use of treating intrauterine and endometrial malignancies/disorders. In one embodiment of the patent a blocking device is described in which the cervical os is occluded to prevent leakage of substances intended for electroporation into the inner uterine lining. This blocking device lacks any conductive surface or layer to electroporate any aspect of the cervix. A detailed analysis of the documents reveals no mention of intent or purpose of the device for specific use in cervical malignancies and disorders. Our intrauterine balloon device is merely a negative ground lead and not the critical therapeutic device directly delivering pharmacologic agents to the cervix. Even greater distinction can be made based on the fact U.S. Pat. No. 5,749,845 is incapable of electroporating neither the inner or outer surface of the cervix. Granted the inventors in U.S. Pat. No. 5,749,845 use the words uterus corpus and cervi. However, those familiar with the field will easily and quickly recognize a tremendous difference in the pathogenesis and diseases of the cervix versus those found in the uterus. Individuals familiar with the field of gynecologic oncology readily attest the cervix and uterus are anatomically two distinct entities composed of different tissues and cells in addition to manifesting different pathologic diseases.

In U.S. Pat. No. 5,816,248 a device with electroporative capabilities is described. This device was capable of inducing constriction of the anterior vaginal wall to tighten the urogenital floor and treat urinary incontinence. Again this patent does not mention or describe any device or method in which therapeutic agents may be introduced to a cervix via electroporation.

In U.S. Pat. No. 5,389,069 a remote electroporation device is extensively covered. While this device has general applications that might be far reaching, it requires invasive penetration with a trochar or long needle to instill the target site prior to electroporation. This apparatus and technique is dramatically different from our device, since the cervical cup does not require invasive injection nor induce mechanical trauma to the cervix in order to deliver pharmaceutical preparations prior to the electroporation step.

### SUMMARY OF THE INVENTION

The invention relates in general to drug delivery and in particular to the field of electroporation, phonophoresis and magnetophoresis and iontophoresis. The invention is composed of two key elements. The first is a cervical cup or sleeve that when placed around the cervix is able to effect a tight seal and intimate association with the internal and external surfaces of the cervix. The device fits in a similar fashion to a contraceptive barrier diaphragm. Those familiar with the field of gynecologic oncology will know that the cervix of a patient can be easily exposed and examined with a speculum. By exposing the cervix with a speculum, the cervical cup placement may be confirmed with visual inspection. Multiple cup sizes may be manufactured and a trial of various shapes and sized cups may be used to provide the closest fit.

The unique property of the cervical cup is that it may serve as both the drug delivery device and the negative lead for electroporation. The cup may be manufactured of any efficient material required to electroprate reagents and therapeutics into cervical cells. The inner most layer in direct contact with the cervix may be fenastrated and enlaced with fine channels that ultimately communicate into one large channel accessible by a syringe to inject therapeutic agents in order to coat or bathe the cervical surface prior to electroporation.

Prior to engaging the cup onto the cervix, the therapeutic agent in a liquid powder, gel, vapor or gas form may also be applied to the conductive surface to come in contact with the cervix. Alternatively, the therapeutic agents may be topically applied or injected onto the cervix prior to cup placement. Additionally, if the cup as in FIGS. 1-9 is used, the therapeutic agent may be injected after the cup is seated on the cervix. Once this cup is properly seated on the cervix (such that the central tunnel traverses through the endocervical canal and the lateral edges of the cup completely cover the entire exterior surface of the cervix) in a fashion similar to a diaphragm, the second key element may be engaged.

The second key element is an expandable balloon catheter that when deflated traverses the inner opening of the cervical cup and is advanced into the uterine cavity. (The cervix may require gentle dilation prior to fitting of cup and advancing of balloon catheter. This can be easily achieved with serial dilation commonly employed in standard gynecologic procedures such as dilation and curettage.) The balloon is constructed of an elastic balloon with an outer skin made of an electrically conductive material. The balloon is deployed by injecting air/liquid/gel via a syringe connected to a port and tube which communicates with the balloon. When deployed the balloon is capable of molding into any variety of uterine cavity shapes in addition to forming an internal negative lead used for enhanced in vivo electroporation. The conductive material is connected to an insulated wire which extends atraumatically from the uterine cavity through the cervical canal and vagina to make final contact with the positive lead for electroporation purposes. This positive lead may connect to any electroporation device emitting tailored and specific wave forms, pulses, time course, and/or patterns optimal for the intended treatment. Additionally, the internal lead shaft may be used as a source of counter-traction for closer contact of the cervical cup with the cervix by clamping the shaft with a forcep proximal to the cervical cup. The internal lead or the cervical cup may be modified so that only a limited surface area is coated with conductive material. This would allow the placement of the internal lead and cervical cup in strategic areas that allow tailored electrophoresis fields to treat only diseased regions of the cervix. Note that the first and second lead may be switched in polarity to achieve optimal drug delivery. The two leads, however, must be placed in a bipolar arrangement.

Alternatively enhanced electroporation into the endocervical canal can be achieved by using a modified cervical cup (FIGS. 7-9) that contains the positive lead on the outer circumference and negative lead on the inner circumference on the cup alone. This setup would yield an electric field that emanates axially throughout the endocervical canal during electroporation and would not require the use of a positive intrauterine lead.

### BRIEF DESCRIPTION OF THE DRAWINGS

FIG. 1. Shows the cervical electroporation cup (similar to a diaphragm) with inner layer composed of an electrically conductive material. Though not pictured the cup may be attached to a rod or stabilizing shaft that does not occlude the inner cylindrical opening to obtain better manual control over placement of the device. The innermost layer may also be inter-laced or embedded with material containing pharmaceuticals or a series of microfenestrations and pores communicating with a single channel and port, whereby therapeutic agents may be injected using a syringe.

FIG. 2. Shows the intrauterine balloon lead in a collapsed state. (Not deployed.)

FIG. 3. Shows the intrauterine balloon lead deployed/inflated.

FIG. 4. Shows both cervical cup and intrauterine balloon deployed in a standard therapeutic fashion. Intrauterine balloon lead is shown as having traversed through the inner lumen of the cervical cup and deployed upon reaching the uterine cavity.

FIG. 5. Shows an alternate representation of FIG. 4 depicting system fully deployed in vivo with balloon completely inflated within the uterus and cervical surfaces completely covered by the electroporation cup. Uterus and cervix are drawn as a vertical section through the human female reproductive and genital tract.

FIG. 6. Shows a conceptual representation of device in FIGS. 4 and 5 and the direction in which negatively charged molecules or therapeutic agents would traverse (in small arrows) if electroporated with the leads used in the manner exhibited in FIGS. 4 and 5.

FIG. 7a. Shows the cervical electroporation cup modified to contain two independent conductive surfaces attached in a bipolar fashion for enhanced electroporation into the endocervical canal.

FIG. 7b. Same object as in FIG. 7a, but shown as a top view.

FIG. 7c. Same as FIG. 7b, but shown as a cross-sectional view.

FIG. 8. Shows an alternate representation of FIG. 7 depicting system fully deployed in vivo with balloon completely inflated within the uterus and cervical surfaces completely covered by the electroporation cup. Uterus and cervix are drawn as a vertical section through the human female reproductive and genital tract.

FIG. 9. Shows a conceptual representation of device in FIGS. 7 and 8 and the direction in which negatively charged molecules or therapeutic agents would traverse (in small arrows) if electroporated with the leads used in the manner exhibited in FIG. 8.

### DESCRIPTION OF PREFERRED EMBODIMENTS

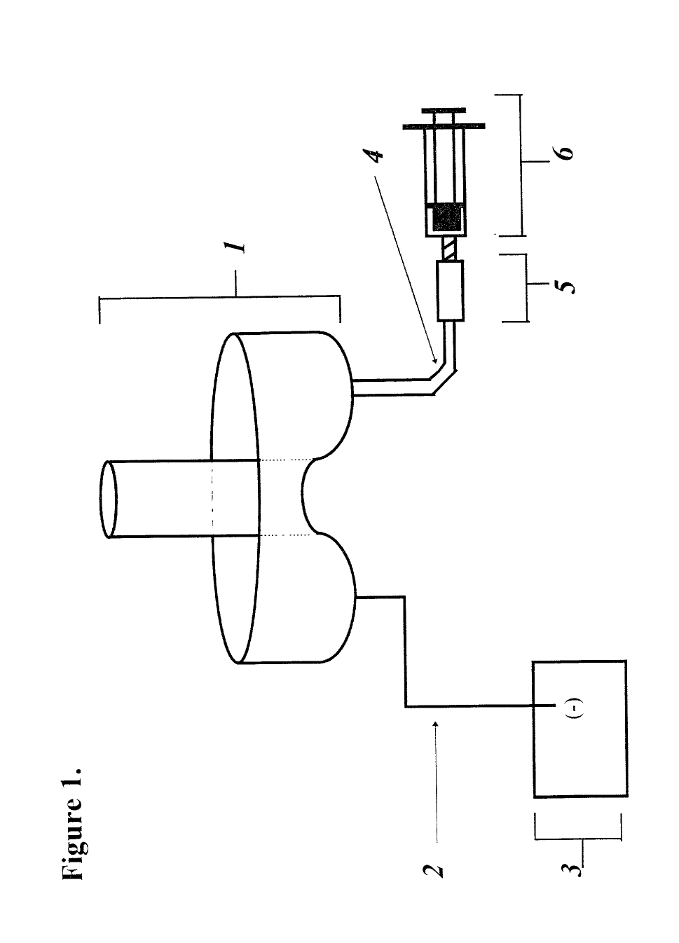

FIG. 1: The cervical cup 1 contains an inner layer comprised of an electrically conductive surface that is connected to an insulated wire 2, which passes atraumatically out of the body and connects to a power source or electroporation device 3. The inner layer of 1 may also be concomitantly interlaced with a series of channels that ultimately communicate to a tube 4 that passes atraumatically out of the body and ultimately connects to a port 5 and syringe 6 to be used as an injection device for therapeutics in liquid/gel/vapor/gas form.

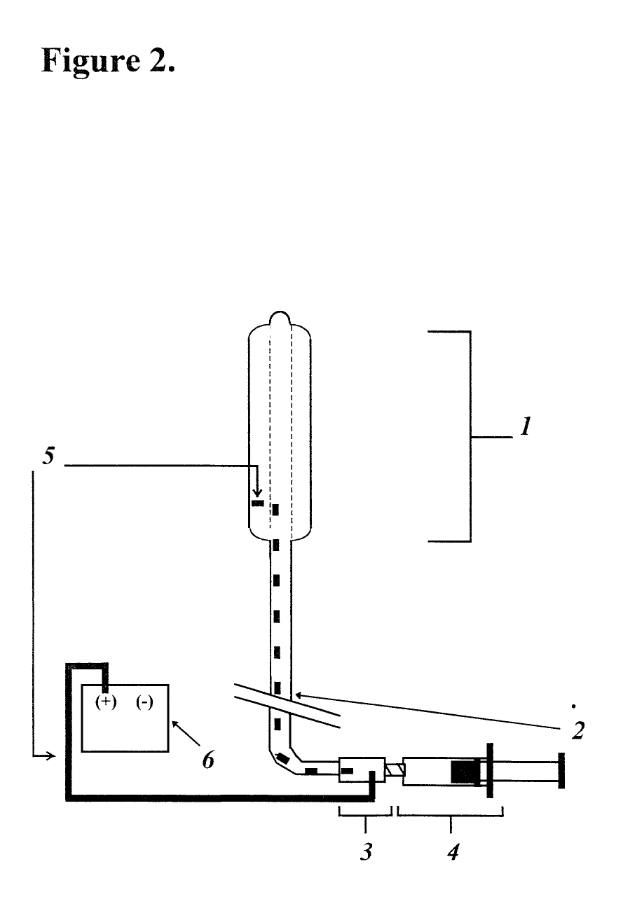

FIG. 2. The internal lead is charged opposite the cervical cup used in FIG. 1. 1 is the expandable balloon that has an outer electrically conductive surface that comes in direct adposition to the inner uterine lining when deployed by injecting liquid, gel or air via the syringe 4 traversing through a syringe port 3 that then attaches to a rubber tubing 2, which ultimately communicates to the intrauterine balloon 1. A seamless insulated wire 5 connects the conductive surface on 1 to an electroporation device or power supply 6.

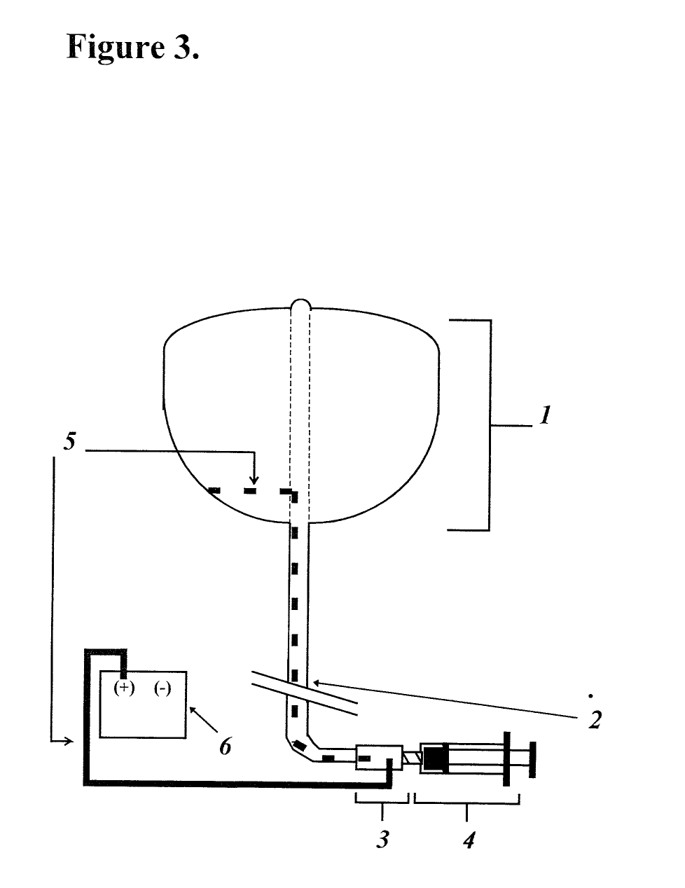

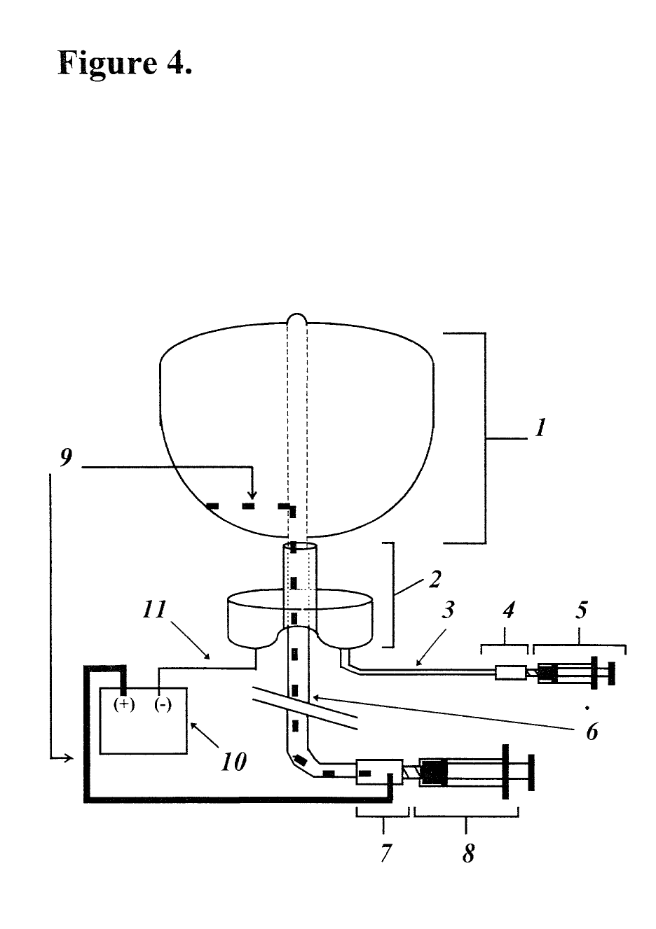

FIG. 3. The same internal lead as described in FIG. 4, however, the balloon device 1 is deployed upon injection with air/liquid/gel from syringe 4. Again 3 is the syringe port, 4 is the syringe and 2 is the tube that allows communication of balloon 1 to syringe 4 and also houses an insulated wire 5 which connects the conductive surface on balloon 1 to the power source/electroporation device 6.

FIG. 4. Intrauterine balloon 1 and cervical cup 2 deployed in a standard therapeutic fashion with the balloon traversing through the inner opening of the cervical cup 2 and the balloon 1 fully inflated. The cup 1 is connected by an insulated atraumatic wire 11 connecting to a power source or electroporation device 10. Again the inner layer of 2 may also be concomitantly interlaced with a series of channels that ultimately communicate to a tube 3 that passes atraumatically out of the body and ultimately connects to a port 4 and syringe 5 to be used as an injection device for therapeutics in liquid/gel/vapor/gas form. Furthermore connected to the intrauterine balloon is a tube 6 that allows communication with the port 7 and syringe 8 which deploys the balloon. Wire 9 connects conductive surface on balloon 1 with power source or electroporation device 10. Wire 11 connects conductive surface of cup 1 to negative lead on power source 10.

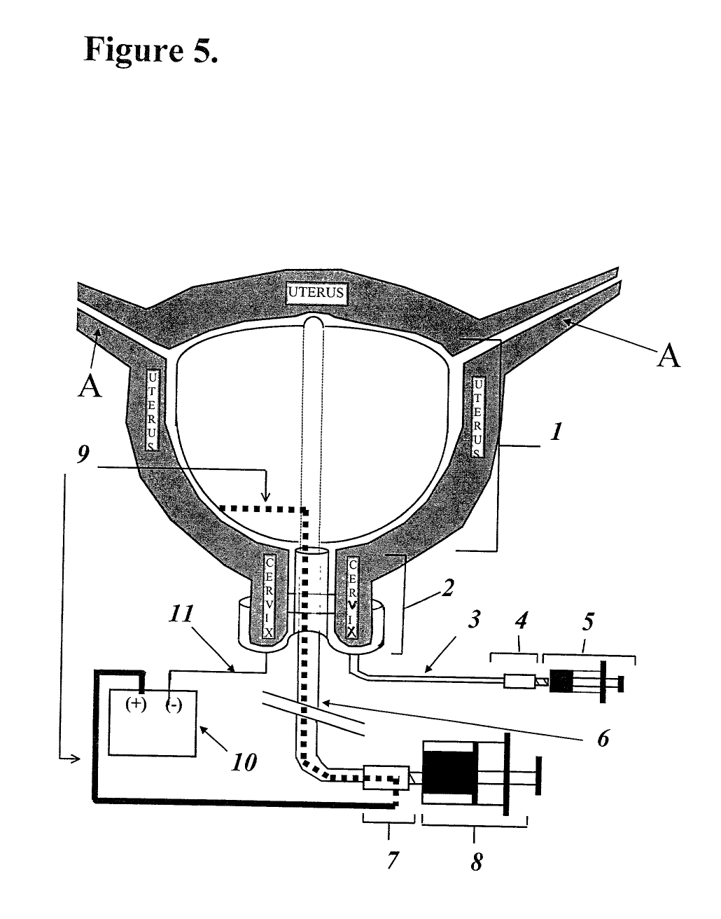

FIG. 5. The same instrument set up as in FIG. 4, however, the apparatus is depicted in vivo and as a vertical section of through the human female reproductive tract with the uterus and cervix as labeled and the fallopian tubes A. Details of numbered objects are the same as described in FIG. 4.

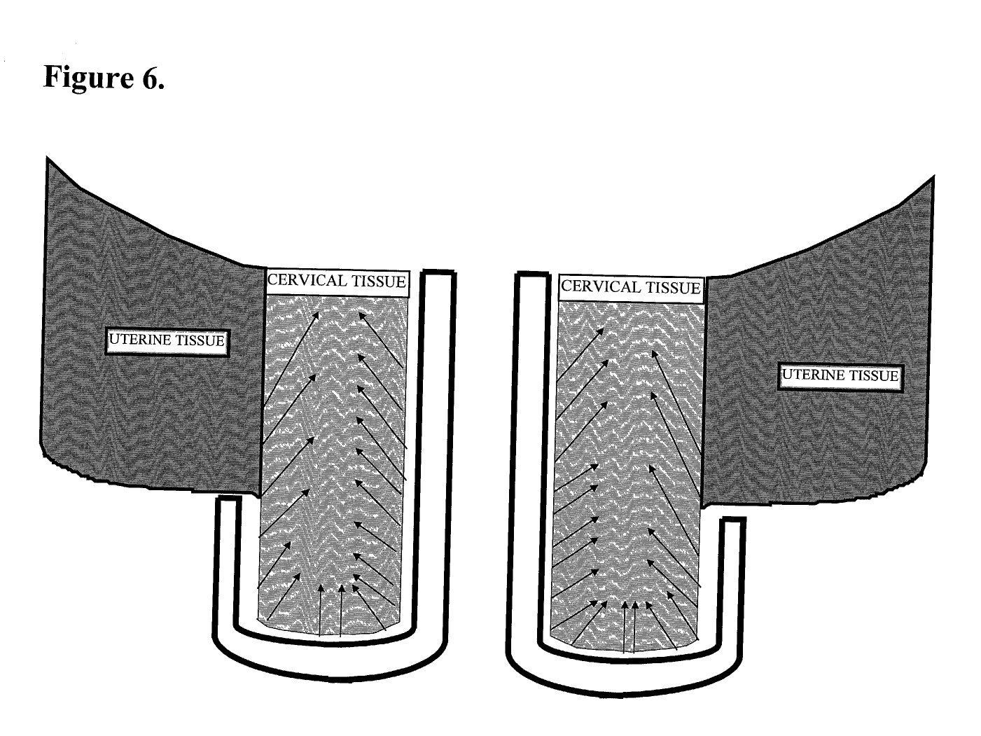

FIG. 6. A conceptual drawing of device stated in FIG. 5. For clarity, device is depicted as a cross-sectional representation of the cervical cup minus the wires, tubing, syringe and power source or electroporation device. When the device used in FIG. 5 is triggered to deliver an electrical pulse, it will create electroporation of substances in the direction of the small arrows indicated. The polarity on surfaces of the cervical cup and intrauterine lead may be reversed in polarity, but typically opposite to one another, in order to enhance delivery of positively charged therapeutic agents.

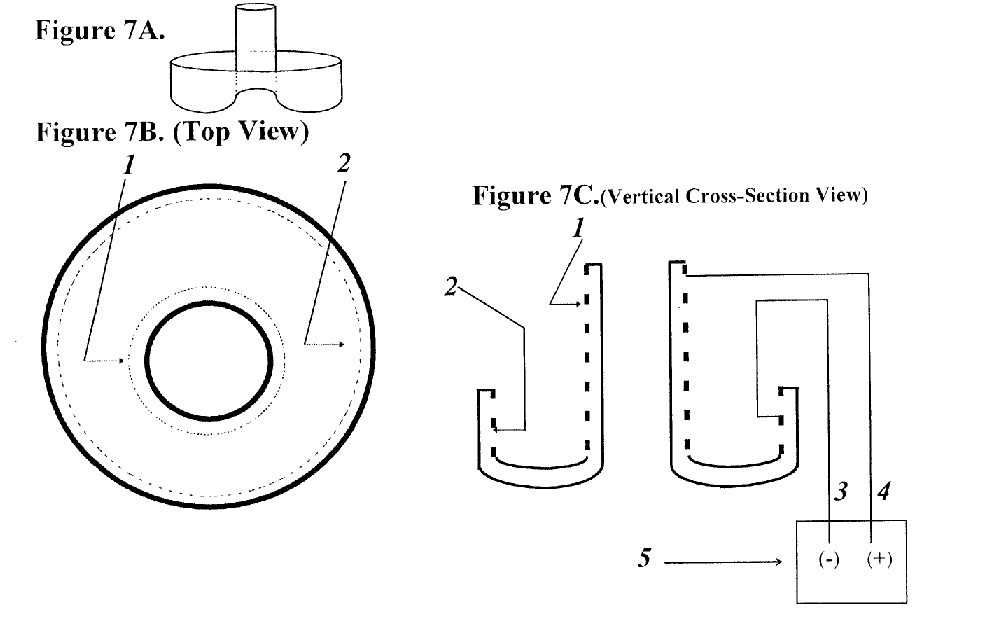

FIG. 7: The same device as in FIG. 1, however, the inner surface of the cervical cup is composed of two separate electrically conductive surfaces surrounding the inner cylinder 1 and the inner aspect of the outer rim 2. Surface 1 would be in contact with the endocervical canal, whereas 2 is in direct contact with the outermost lateral aspect of the cervix. Again 3 being an atraumatic insulated wire that connects conductive surface 2 to either an electroporation device or power source 5. 4 is another insulated atraumatic wire which connects the conductive surface 1 to the same electroporation device or power source 5, but typically in opposite polarity to conductive surface 2. Polarities of conductive surfaces 1 and 2 may be switched to suit the direction of electroporation according to the charge of the agent to be delivered. Though not depicted, a tube connecting microfenestrated and embedded channels in any specific region of the cervical cup to a delivery syringe may be fashioned for delivery of therapeutic agents prior to electroporation. Dashed lines in FIG. 7B (top-view) and 7C (vertical cross-section view) represent electrically conductive surfaces.

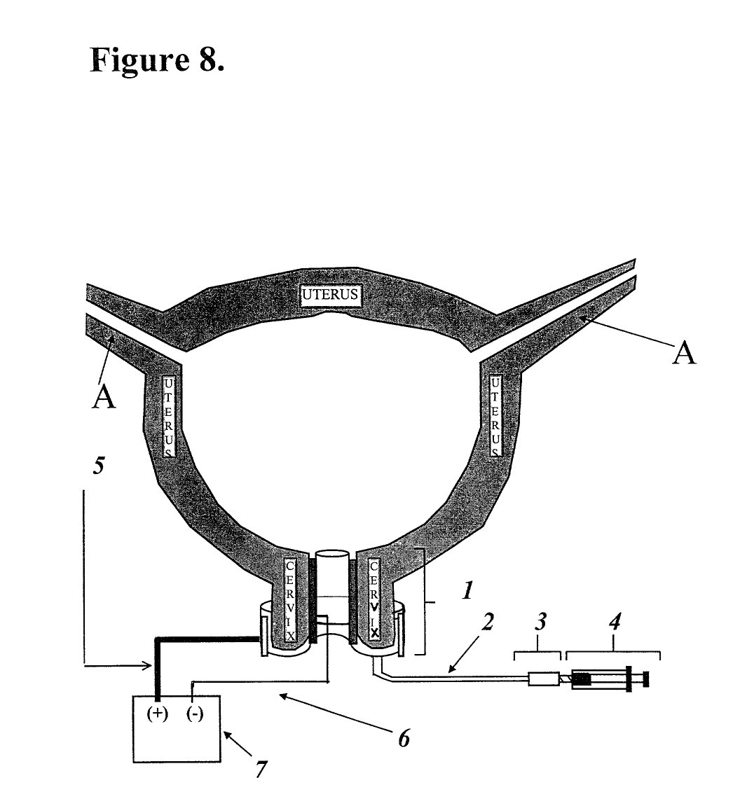

FIG. 8. Device in FIG. 7 shown seated in vivo with uterine and cervical tissues as indicated and shaded in medium gray tone. Fallopian tubes are labelled as A. Inner circumference conductive surface in dark gray tone is connected to the negative wire 6 and lead on power supply/electroporation device 7. Opposing conductive surface in light gray tone is connected by positive wire 5, which connects to positive lead on same power supply/electroporation device 7.

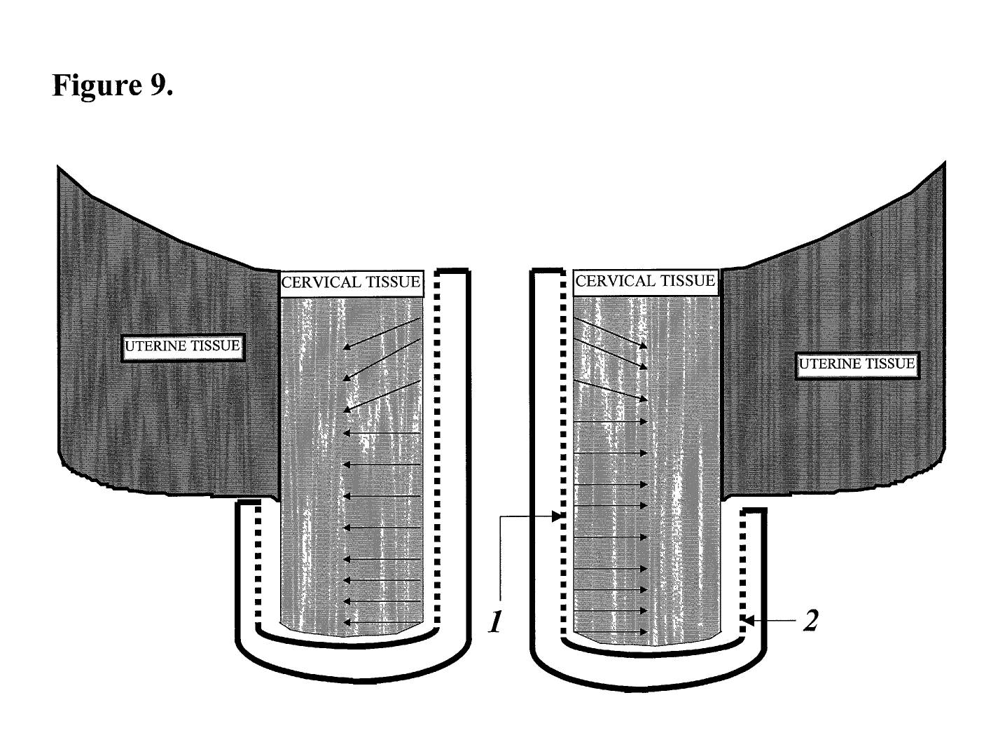

FIG. 9. A conceptual drawing of device stated in FIGS. 7 and 8. For clarity, device is depicted as a cross-sectional representation of the cervical cup minus the wires, tubing, syringe and power source or electroporation device. Conductive surfaces 1 and 2 depicted as dotted lines are directly facing opposite one another and when activated (i.e. electroporation device or power source is triggered to deliver a pulse or current) will create electroporation of substances in the direction of the small arrows indicated. The polarity of surfaces 1 and 2 may be adjusted to suit the charge of the therapeutic agent being introduced into tissue.

Embodiments:

A further embodiment entails using the proposed inventions for electroporating various agents into a normal or diseased cervix for cervical ripening during prolonged pregnancies, especially in females refractory to conventional therapeutics such as pitocin. Use of the internal intrauterine lead in this circumstance will not be used in conjunction with the cervical cup. Electrical pulses and currents generated from the electroporation device or power source would be tailored for maximal delivery of therapeutic agent with zero to negligible effect(s) on the fetus.

In a further embodiment of the invention the cup may be used solely with lipofection and drug agents added and held in place for extended time with any efficient and atraumatic clamp or holder. Moreover greater extended therapy (brachytherapy) may be achieved by creating an internal holder that loops in the internal os and secures the cup snug against the cervix.

In a further embodiment one may use the electroporation device without any agent and use the effects of electrical current on the cervix for increasing porosity of the cervix for systemic drugs. As electroporation fields in themselves have also exhibited a bystander effect. The electroporation field in itself may be used to affect cures for various diseases and disorders of the cervix.

In yet another embodiment, the distal end of the cervical cup, that traverses through the cervical os and canal, may be fashioned of a resilient and flexible material with memory, that when shrouded in a holder is held in a cylindrical shape. When the central cylinder is advanced through the endocervical canal, the shroud may be released and removed allowing the distal cylinder to fold backwards on itself into the original molded shape enclosing the junction of the inner uterine lining and the beginning of the cervical outlet. This design modification would allow the electroporation of the internal intrauterine outlet leading into the cervical canal. The distal flexible end of the inner cylinder may also be designed to take any form suitable for electroporation of the intrauterine aspect of the cervix.

In yet another embodiment the leads depicted in FIGS. 1 and 7 may be reversed in polarity (but still maintaining bipolar configuration) in order to force positively charged substances into cells. Both magnitude and charge of bipolarity between positive and negative leads may be adjusted to increase efficiency of therapeutic agent delivery into cervical tissue.

Additionally the negative internal lead may be abandoned all together and multiple external patch leads or shorts with a conductive surface may be used to create tailored electrical fields to focus the direction of electroporation toward specific regions of diseased cervix.

In a further embodiment the system may utilize alternate forms of energy transfer in place of electroporation/iontophoresis. These forms include, but are not limited to, phonophoresis and magnetophoresis.

In another embodiment the inductive circuit can be designed as a resonant circuit tuned to the radio frequency range (>9 KHz) capable of producing electrical current when exposed to an oscillating magnetic field tuned at the same frequency. (Stray magnetic field interference is mitigated with this design.) The power supply may also have an AC/DC converter to exclusively direct current to pass through the positive and negative leads.

1. A pharmacologic/gene therapeutic/drug agent delivery system by in vivo and in situ electroporation into a mammalian cervix:
a. applied for the use of adjuvant therapy after standard ablative or resection techniques
b. applied for the use of preventative treatment of premalignant lesions
c. applied for the use of treatment of benign or malignant lesions
d. applied for the use of treating other disorders of the cervix such as delivery of ripening agents to allow vaginal delivery of a child.
e. applied for the use of delivering agents to enhance strength or growth to a cervix previously resected of premaligant or malignant tissue requiring further growth and regeneration in anticipation and preparation for pregnancy.

2. A modifiable positive intrauterine lead to allow tailored directional electroporation into specific regions of the mammalian cervix:
a. utilizing an intrauterine lead similar to the one stated above, but with selectively placed regions of conductive surfaces to provide directed fields of electroporation specifically tailored to penetrate cervical regions containing malignant or premalignant lesions.

3. A modifiable cervical cup containing positive and/or negative leads capable of conforming with the outer (ectocervix) and/or inner (endocervical) mammalian cervix:
a. for use in delivering therapeutic agents exclusively to the mammalian endocervical canal
b. for use in delivering therapeutic agents exclusively to the outer cervical lining of the mammalian cervix
c. with conductive surfaces that may be changed in polarity, voltage, shape or size to maximize delivery therapeutic of agents
d. that can be modified to encompass a greater surface area covering the cervix and surrounding (paracervical) vaginal mucosa to treat diffuse lower tract genitourinary disease
e. that can be modified to have solid or hollow needles to enhance electroporation or injection of therapeutic drugs into solid genitourinary tumor or tissue.
